(19) United States
(12) Patent Application Publication
(10) Pub. No.: US 2017/0226029 A1
(43) Pub. Date: Aug. 10, 2017

(54) METHODS OF PRODUCING ETHYLENE AND SYNTHESIS GAS BY COMBINING THE OXIDATIVE COUPLING OF METHANE AND DRY REFORMING OF METHANE REACTIONS

(71) Applicant: SABIC Global Technologies B.V, Bergen op Zoom (NL)

(72) Inventors: Aghaddin Mamedov, Sugar Land, TX (US); David West, Sugar Land, TX (US); Vemuri Balakotaiah, Sugar Land, TX (US); Sagar Sarsani, Sugar Land, TX (US); Wugeng Liang, Sugar Land, TX (US)

(73) Assignee: SABIC Global Technologies B.V., Bergen op Zoom (NL)

(21) Appl. No.: 15/518,993

(22) PCT Filed: Dec. 9, 2015
(86) PCT No.: PCT/US15/64628

§ 371 Date: Apr. 13, 2017

Related U.S. Application Data
(60) Provisional application No. 62/089,344, filed on Dec. 9, 2014.
(60) Provisional application No. 62/089,348, filed on Dec. 9, 2014.

(51) Int. Cl. C07C 2/84; B01J 8/02; B01J 23/34; B01J 35/00; C01B 3/38; B01J 21/08

(52) U.S. Cl. CPC C07C 2/84; C01B 3/38; B01J 21/08; B01J 23/34; B01J 35/0006; B01J 8/0278; C01B 2203/0238; C01B 2203/0838; C01B 2203/1082; C01B 2203/1041; C01B 2203/1241; C07C 2523/04; C07C 2523/34; C07C 2523/30; C07C 2523/10; C07C 2521/08

(57) ABSTRACT

Disclosed is a method for production of synthesis gas and ethylene by a combined oxidative coupling and dry reforming of methane process. Heat generated from the oxidative coupling of methane can be used to drive the endothermic dry reforming of methane reaction.

### CROSS REFERENCE TO RELATED APPLICATIONS

This application claims benefit to U.S. Provisional Patent Application No. 62/089,344, titled “METHOD FOR CONVERTING METHANE TO ETHYLENE AND IN SITU TRANSFER OF EXOTHERMIC HEAT”, filed Dec. 9, 2014 and U.S. Provisional Patent Application No. 62/089,348 titled “METHODS OF PRODUCING ETHYLENE AND SYNTHESIS GAS BY COMBINING THE OXIDATIVE COUPLING OF METHANE AND DRY REFORMING OF METHANE REACTIONS”, filed Dec. 9, 2014. The entire contents of the referenced applications are incorporated by reference without disclaimer.

### BACKGROUND OF THE INVENTION

A. Field of the Invention

The invention generally concerns methods of producing C₂ hydrocarbons and synthesis gas. In particular, the methods include simultaneously producing ethylene and synthesis gas from methane, oxygen and carbon dioxide with a controlled heat transfer process.

B. Description of Related Art

Ethylene is typically used to produce a wide range of products, for example, break-resistant containers and packaging materials. For industrial scale applications ethylene is currently produced by heating natural gas condensates and petroleum distillates, which include ethane and higher hydrocarbons, and the produced ethylene is separated from the product mixture using gas separation processes.

Ethylene can also be produced by oxidative coupling of the methane as represented by the following equations:

2CH₄+O₂→C₂H₄+2H₂O ΔH=−34 kcal/mol (I)

2CH₄+1/2O₂→C₂H₄+H₂O ΔH=−21 kcal/mol (II)

Oxidative conversion of methane to ethylene is exothermic. Excess heat produced from these reactions can push conversion of methane to carbon monoxide and carbon dioxide rather than the desired C₂ hydrocarbon product:

CH₄+1.5O₂→CO+2H₂O ΔH=−103 kcal/mol (III)

CH₄+2O₂→CO₂+2H₂O ΔH=−174 kcal/mol (IV)

The excess heat from the reactions in Equations (III) and (IV) further exasperate this situation, thereby substantially reducing the selectivity of ethylene production when compared with carbon monoxide and carbon dioxide production.

Additionally, while the overall oxidative coupling of methane (OCM) is exothermic, catalysts are used to overcome the endothermic nature of the C-H bond breakage. The endothermic nature of the bond breakage is due to the chemical stability of methane. Methane is chemically stable molecule due to the presence of its four strong tetrahedral C-H bonds (435 kJ/mol). When catalysts are used in the oxidative coupling of methane, the exothermic reaction can lead to a large increase of catalyst bed temperature and uncontrolled heat excursions that can produce agglomeration on the catalyst. This leads to catalyst deactivation and a further decrease in ethylene selectivity. Furthermore, the produced ethylene is highly reactive and can form unwanted and thermodynamically favored oxidation products at too high of oxygen concentrations.

U.S. Patent Application Publication Nos. 2014/0121433 to Cizeron et al.; 2013/0023709 to Cizeron et al., and 2013/0165728 to Zurcher et al., describe attempts to control the exothermic reaction of the oxidative coupling of methane by using alternating layers of selective OCM catalysts. Other processes attempt to control the exothermic reaction through the use of fluidized bed reactors and/or to use steam as a diluent. These solutions are costly and inefficient. Further, a large amount of water is required to absorb the heat of the reaction.

### SUMMARY OF THE INVENTION

A solution to the above described problems has been discovered. In particular, the solution resides in combining the exothermic oxidative coupling of methane reaction with the endothermic reaction of dry reforming of methane to produce ethylene and synthesis gas (also known as “syngas”) while also transferring any excess heat to inert material. Dry reforming of methane is represented by equation (V):

CH₄+CO₂→2CO+2H₂ ΔH+60 kcal/mol (V)

Dry reforming of methane refers to the production of carbon monoxide and hydrogen gas from methane and carbon dioxide in the absence of steam or water. By combining the oxidative coupling of methane and dry reforming of methane reactions, the overall reaction of the present invention can be represented as follows:

5CH₄+O₂+CO₂→2C₂H₄+2CO+4H₂+2H₂O ΔH−198 kcal/mol (VI)

This combination allows for increased ethylene selectivity while also reducing the costs associated with syngas production. The use of CO₂ as an oxidant can reduce the consumption of expensive oxygen per mole of converted methane when compared with the oxidative coupling of methane using oxygen as the sole source of oxidant. The methods also substantially eliminate the production of unwanted byproducts such as carbon dioxide by directly converting produced carbon dioxide to synthesis gas. Furthermore, the methods avoid the deactivation of catalysts. Without wishing to be bound by theory, it is believed that the development of hot spots within catalyst beds are controlled, as the heat generated during the exothermic reaction of methane and oxygen not used for the endothermic methane reforming reaction is removed by the inert material, thereby extending the life of the catalysts.

In one particular aspect of the invention, a method of producing ethylene and synthesis gas from a reaction mixture comprising methane (CH₄), oxygen (O₂), and carbon dioxide (CO₂) is described. The method includes contacting the reaction mixture under sufficient conditions to produce a product stream comprising ethylene and synthesis gas. The ethylene is obtained from oxidative coupling of CH₄ and the synthesis gas is obtained from CO₂ reforming of CH₄. Heat produced by the oxidative coupling of CH₄ is (1) transferred to an inert material in an amount sufficient to reduce thermal deactivation of the catalytic material and (2) used in the CO₂ reforming of CH₄. In some instances, the method occurs in a continuous flow reactor, for example, a fixed bed reactor or a fluidized reactor. In the reactant mixture a molecular ratio of CH₄ to CO₂ ranges from 0.3 to 1, 0.5 to 0.8, or 0.6 to 0.7, a molecular ratio of CH₄ to CO₂ range from 1 to 2, and/or a molecular ratio of O₂ to CO₂ ranges from 0.5 to 2, 0.75 to 1.5, or 1 to 1.25. Process conditions to effect production of ethylene and syngas from methane through oxidative coupling and dry reforming of the methane include a temperature of 700 to 900° C. or from 750 to 850° C. and a gas hourly space velocity from 1800 to 80,000 h⁻¹, preferably from 1800 to 50,000 h⁻¹, or more preferably from 1800 to 20,000 h⁻¹. Heat generated during the reaction can be transferred from the inert material to a cooling fluid or medium. Non-limiting examples of the inert material are magnesium oxide (MgO), silicon dioxide (SiO₂), or both. The catalytic material of the invention is one or more catalysts that catalyze the oxidative coupling of methane and/or the dry reforming of methane. In one aspect of the invention, the catalytic material includes manganese (Mn) or a compound thereof, lanthanum (La) or a compound thereof, sodium (Na) or a compound thereof, cesium (Cs) or a compound thereof, calcium (Ca) or a compound thereof, and any combination thereof. Non-limiting examples of the catalytic material include La/Mg, Na—Mn—La₂O₃/Al₂O₃, Na—Mn—O/SiO₂, Na₂WO₄—Mn/SiO₂, or any combination thereof. In another aspect of the invention, the catalytic material is mixed with or dispersed in the inert material, or both. A weight ratio of catalytic material to the inert material ranges from 5 to 30, preferably from 5 to 20, or more preferably from 7 to 15. In an aspect of the invention, the temperature of the catalytic material does not exceed it deactivation temperature, such as, for example, 800 to 900° C. In a particular aspect, the temperature of the catalytic material does not exceed its deactivation temperature for about 10 to 20 minutes. In an aspect of the invention, 90% or more of the reactant mixture is converted into ethylene and synthesis gas. The method has a selectivity to ethylene is 30 to 50%. In the method, 75% or more, or more preferably 90% or more of the methane is converted to ethylene and synthesis gas. In some aspects of the invention, the method can further include isolating and/or storing the produced gaseous mixture. The method can further include separating the ethylene from the synthesis gas (such as passing the mixture of ethylene and synthesis gas through multiple gas selective membranes).

In one aspect of the invention, the catalytic material is positioned upstream from the inert material. The catalytic material and the inert material are configured in multiple alternating layers and that the inert layer has a thickness that is greater than the thickness of the catalytic material layer. The catalytic material and/or the inert material can be configured as layers and the thickness of a first inert material layer is greater than the thickness of a first catalytic material layer. In some aspects of the invention, the total number of layers of the catalytic material is equal to x, and the total number of layers of the inert material is equal to x−1, x+1, or x. The total number of layers of the catalytic material ranges from 3 to 50, preferably from 3 to 25, or more preferably from 3 to 5. It should be understood that the catalytic material and inert material can be alternated to produce a desired number of repeating materials. In a particular aspect of the invention, the inert material is a positioned downstream of the catalytic material in a reactor and the catalytic material and inert layers having a desired thickness are repeated until the desired number of inert layers and catalytic material layers are achieved. The thickness and the number of layers can be varied such that the heat produced from the exothermic oxidative coupling reaction is controlled in situ. Changing the thickness of the catalytic material layers and the inert layers allows the heat to be transferred to the walls of the vessel and/or transferred to methane molecules in a controlled manner, thereby extending the life of the catalyst, increasing the conversion of methane, oxygen and carbon dioxide to ethylene and synthesis gas and increasing the selectivity of ethylene production. Due to the control of the heat during the reaction period, the overall oxidation of methane to carbon dioxide is diminished and/or inhibited. Without wishing to be bound by theory, it is believed that the conversion and catalyst temperatures within the layers of catalytic material and the inert layers depend on a dimensionless group referred to as the transverse Pèclet number (P), which is the ratio of the interphase transport time to the convection time. When P is less than about 0.1 (P<0.1), the transport rate between the reactant mixture and the catalyst is high compared to the flow rate of reactants. When P is much greater than 0.1 (P>>0.1), the transport limitation between the fluid and the catalyst limits the temperature rise in the catalyst phase. Depending on the thickness of the layers of both catalytic material and inert material, the magnitude of P within each layer can be controlled. Controlling the magnitude of P for each layer controls the temperature profile in the reactor. When P>0.1 within a catalytic layer, the temperature rise and amount of reaction within such layer is limited, thereby eliminating the extreme rises in temperature. In certain aspects of the invention, the product stream formed from contacting the first catalytic material and/or second catalytic material contacts the third catalytic material and produces ethylene and synthesis gas. The ethylene is obtained from oxidative coupling of CH₄ and synthesis gas is obtained from CO₂ reforming of CH₄. Heat produced by the oxidative coupling of CH₄ is (1) transferred to the first and second inert materials in an amount sufficient to reduce thermal deactivation of the second catalytic material and (2) used in the CO₂ reforming of CH₄.

In the context of the present invention forty-five (45) embodiments are disclosed. In a first embodiment, a method of producing ethylene and synthesis gas from a reactant mixture that includes methane (CH₄), oxygen (O₂) and carbon dioxide (CO₂) is disclosed. The method can include contacting the reactant mixture with a catalytic material to produce a product stream comprising ethylene and synthesis gas, wherein the ethylene is obtained from oxidative coupling of CH₄ and the synthesis gas is obtained from CO₂ reforming of CH₄, wherein heat produced by the oxidative coupling of CH₄ is used in the CO₂ reforming of CH₄. Embodiment 2 is the method of embodiment 1, wherein the catalytic material can include a catalyst, or a mixture of catalysts, that catalyze the oxidative coupling of CH₄ and the CO₂ reforming of CH₄. Embodiment 3 is the method of embodiment 2, wherein the mixture of catalysts includes a first catalyst that catalyzes the oxidative coupling of CH₄ and a second catalyst that catalyzes the CO₂ reforming of CH₄. Embodiment 4 is the method of embodiment 3, wherein the mixture of catalysts includes Na₂O, Mn₂O₃, WO₃, and La₂O₃. Embodiment 5 is the method of any one of embodiments 1 to 4, wherein the ratio of CH₄:O₂:CO₂ in the reactant mixture is 1:0.5:1. Embodiment 6 is the method of embodiment 5, wherein the reaction temperature is 750° C. to 900° C. Embodiment 7 is the method of any one of embodiments 4 to 6, wherein 20% to 60% methane was converted, and the selectivity to ethylene is 30% to 35% and the selectivity to carbon monoxide is 15% to 70%, or 65% to 70%. Embodiment 8 is the method of any one of embodiments 1 to 7, wherein the method occurs in a continuous flow reactor. Embodiment 9 is the method of embodiment 8, wherein the continuous flow reactor is a fixed-bed reactor or a fluidized reactor. Embodiment 10 is the method of any one of embodiments 1 to 9, wherein heat produced by the oxidative coupling of CH₄ is (1) used in the CO₂ reforming of CH₄ and (2) transferred to an inert material in an amount sufficient to reduce thermal deactivation of the catalytic material. Embodiment 11 is the method of embodiment 10, wherein the catalytic material is positioned upstream from the inert material. Embodiment 12 is the method of embodiment 11, wherein heat is transferred from the inert material to a cooling fluid or medium. Embodiment 13 is the method of any one of embodiments 11 to 12, wherein the catalytic material and the inert material are configured in multiple alternating layers, and wherein the total number of layers of the catalytic material is equal to x, and the total number of layers of the inert material is equal to x−1, x+1, or x. Embodiment 14 is the method of embodiment 13, wherein the total number of layers of the catalytic material ranges from 3 to 50, 3 to 25, or 3 to 5. Embodiment 15 is the method of any one of embodiments 13 to 14, wherein the inert layer has a thickness that is greater than the thickness of the catalytic material layer. Embodiment 16 is the method of any one of embodiments 10 to 15 that can include at least a second catalytic material and at least a second inert material, wherein the second catalytic material is positioned downstream from the first inert material, and the second inert material is positioned downstream from the second catalytic material. Embodiment 17 is the method of embodiment 16 that can include at least a third catalytic material that is positioned downstream from the second inert material. Embodiment 18 is the method of any one of embodiments 16 to 17, wherein the first catalytic material is configured as a layer, and the first inert material is configured as a layer having a thickness that is greater than the thickness of the first catalytic material layer. Embodiment 19 is the method of embodiment 18, wherein the second catalytic material is configured as a layer having a thickness that is less than the first inert layer and the second inert material is configured as a layer having a thickness that is greater than the thickness of the second catalytic material layer. Embodiment 20 is the method of embodiment 19, wherein the third catalytic material is configured as a layer having a thickness that is less than the thickness of the second inert material layer. Embodiment 21 is the method of embodiment 19, wherein the third catalytic material is configured as a layer having a thickness that is greater than the thickness of the first inert material layer or that is greater than the thickness of the second inert material layer. Embodiment 22 is the method of any one of embodiments 16 to 21, wherein the product stream contacts the second catalytic material and produces ethylene and synthesis gas, wherein the ethylene is obtained from oxidative coupling of CH₄ and synthesis gas is obtained from CO₂ reforming of CH₄, and heat produced by the oxidative coupling of CH₄ is (1) transferred to the first and second inert materials in an amount sufficient to reduce thermal deactivation of the second catalytic material and (2) used in the CO₂ reforming of CH₄. Embodiment 23 is the method of embodiment 22, wherein the product stream contacts the third catalytic material and produces ethylene and synthesis gas, wherein the ethylene is obtained from oxidative coupling of CH₄ and synthesis gas is obtained from CO₂ reforming of CH₄, and eat produced by the oxidative coupling of CH₄ is (1) transferred to the second inert material in an amount sufficient to reduce thermal deactivation of the third catalytic material and (2) used in the CO₂ reforming of CH₄. Embodiment 24 is the method of any one of embodiments 10 to 12, wherein the catalytic material is dispersed in the inert material. Embodiment 25 is the method of embodiment 24, wherein the ratio, by wt. %, of the catalytic material to the inert material is 5 to 30, 5 to 20, or 7 to 15. Embodiment 26 is the method of any one of embodiments 10 to 25, wherein the inert material is chemically inert. Embodiment 27 is the method of any one of embodiments 10 to 26, wherein the inert material is magnesium oxide, silicon dioxide, quartz, or any combination thereof. Embodiment 28 is the method of any one of embodiments 10 to 27, wherein the temperature of the catalytic material does not exceed its deactivation temperature for more than 20 minutes. Embodiment 29 is the method of any one of embodiments 10 to 27, wherein the temperature of the catalytic material does not exceed its deactivation temperature. Embodiment 30 is the method of any one of embodiments 28 to 29, wherein the deactivation temperature is 800° C. to 900 to ° C. Embodiment 31 is the method of any one of embodiments 1 and 8 to 30, wherein the catalytic material comprises a catalyst that catalyzes the oxidative coupling of CH₄. Embodiment 32 is the method of any one of embodiments 1 and 8 to 30, wherein the catalytic material comprises a catalyst that catalyzes the CO₂ reforming of CH₄. Embodiment 33 is the method of any one of embodiments 1 and 8 to 30, wherein the catalytic material comprises a catalyst, or a mixture of catalysts, that catalyzes the oxidative coupling of CH₄ and the CO₂ reforming of CH₄. Embodiment 34 is the method of any one of embodiments 1 to 33, wherein the catalyst comprises manganese or a compound thereof, lanthanum or a compound thereof, sodium or a compound thereof, cesium or a compound thereof, calcium or a compound thereof, and any combination thereof. Embodiment 35 is the method of embodiment 34, wherein the catalyst comprises La/MgO, Na—Mn—La₂O₃/Al₂O₃, Na—Mn—O/SiO₂, Na₂WO₄—Mn/SiO₂, or any combination thereof. Embodiment 36 is the method of any one of embodiments 1 and 8 to 35, wherein the molecular ratio of CH₄ to O₂ in the reactant mixture is 0.3 to 1. Embodiment 37 is the method of any one of embodiments 1 and 8 to 36, wherein the molecular ratio of CH₄ to CO₂ in the reactant mixture is 1 to 2. Embodiment 38 is the method of any one of embodiments 1 and 8 to 37, wherein the molecular ratio of O₂ to CO₂ in the reactant mixture is 0.5 to 2. Embodiment 39 is the method of any one of embodiments 1 and 8 to 38, wherein the method occurs at a temperature range of 700 to 900° C. Embodiment 40 is the method of any one of embodiments 1 and 8 to 39, wherein the weight hourly space velocity is from 1800 to 80,000h⁻¹, from 1800 to 50,000h⁻¹, or 1800 to 20,000 h⁻¹. Embodiment 41 is the method of any one of embodiments 1 to 40, wherein at least 90% of the reactant mixture is converted into ethylene and synthesis gas. Embodiment 42 is the method of any one of embodiments 1 to 41, wherein the selectivity to ethylene is 30 to 50%. Embodiment 43 is the method of any one of embodiments 1 to 42, wherein methane conversion is at least 75%, or at least 90%. Embodiment 44 is the method of any one of embodiments 1 to 43, wherein the produced ethylene and synthesis gas are separated from one another. Embodiment 45 is the method of any one of embodiments 1 to 44, wherein the inert material has substantially no catalytic active for oxidative coupling of methane.

The following includes definitions of various terms and phrases used throughout this specification.

The terms “about” or “approximately” are defined as being close to as understood by one of ordinary skill in the art, and in one non-limiting embodiment the terms are defined to be within 10%, preferably within 5%, more preferably within 1%, and most preferably within 0.5%.

The term “substantially” and its variations are defined as being largely but not necessarily wholly what is specified as understood by one of ordinary skill in the art, and in one non-limiting embodiment substantially refers to ranges within 10%, within 5%, within 1%, or within 0.5%.

The terms “inhibiting” or “reducing” or “preventing” or “avoiding” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the words “a” or “an” when used in conjunction with the term “comprising” in the claims or the specification may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The methods of the present invention can “comprise,” “consist essentially of,” or “consist of” particular ingredients, components, compositions, etc. disclosed throughout the specification. With respect to the transitional phase “consisting essentially of,” in one non-limiting aspect, a basic and novel characteristic of the methods is the ability to produce ethylene and synthesis gas from methane, oxygen and carbon dioxide.

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description.

### BRIEF DESCRIPTION OF THE DRAWINGS

FIG. 1 depicts a schematic of a system of the present invention for the production of ethylene and synthesis gas.

FIG. 2 depicts a schematic of a second system of the present invention for the production of ethylene and synthesis gas.

FIG. 3 is a graphical depiction of temperature versus length of reactor for the system depicted in FIG. 2.

FIG. 4 depicts a schematic of a third system of the present invention for the production of ethylene and synthesis gas.

FIG. 5 is a graphical depiction of temperature versus length of reactor for the system depicted in FIG. 4.

FIG. 6 depicts a schematic of a fourth system of the present invention for the production of ethylene and synthesis gas.

### DETAILED DESCRIPTION OF THE INVENTION

The currently available processes to produce ethylene often result in catalyst deactivation through agglomeration of material on the catalyst surface (coking) and runaway heat due to the heat generated from the highly exothermic reaction between oxygen and methane. This can lead to inefficient ethylene production as well as increased costs associated with its production.

A discovery has been made that controls the generated heat and avoids the catalyst deactivation described above. The discovery is based on a method to produce ethylene and synthesis gas from a reactant mixture containing methane, oxygen and carbon dioxide. The method includes contacting the reactant mixture with a catalytic material to produce a product stream containing ethylene and synthesis gas, where the ethylene is obtained from oxidative coupling of CH₄ and the synthesis gas is obtained from CO₂ reforming of CH₄. The heat produced by the oxidative coupling of CH₄ is (1) transferred to an inert material in an amount sufficient to reduce thermal deactivation of the catalytic material and (2) used in the CO₂ reforming of CH₄.

These and other non-limiting aspects of the present invention are discussed in further detail in the following sections.

### A. Reactants

The reactant mixture in the context of the present invention is a gaseous mixture that includes, but is not limited to, a hydrocarbon or mixtures of hydrocarbons, carbon dioxide and oxygen. The hydrocarbon or mixtures of hydrocarbons can include natural gas, liquefied petroleum gas containing of C₂-C₅ hydrocarbons, C₆+heavy hydrocarbons (e.g., C₆ to C₂₄ hydrocarbons such as diesel fuel, jet fuel, gasoline, tars, kerosene, etc.), oxygenated hydrocarbons, and/or biodiesel, alcohols, or dimethyl ether. In a preferred aspect, the hydrocarbon is methane. Oxygen used in the present invention can be air, oxygen enriched air, oxygen gas, and can be obtained from various sources. Carbon dioxide used in the present invention can be obtained from various sources. In one non-limiting instance, the carbon dioxide can be obtained from a waste or recycle gas stream (e.g. from a plant on the same site, like for example from ammonia synthesis) or after recovering the carbon dioxide from a gas stream. A benefit of recycling such carbon dioxide as a starting material in the process of the invention is that it can reduce the amount of carbon dioxide emitted to the atmosphere (e.g., from a chemical production site). The reactant mixture may further contain other gases, provided that these do not negatively affect the reaction. Examples of such other gases include nitrogen and hydrogen. The hydrogen may be from various sources, including streams coming from other chemical processes, like ethane cracking, methanol synthesis, or conversion of methane to aromatics. The reactant mixture is substantially devoid of water or steam. In a particular aspect of the invention the gaseous feed contains 0.1 wt. % or less of water, or 0.0001 wt. % to 0.1 wt. % water. In the reactant mixture a molecular ratio of CH₄ to O₂ ranges from 0.3 to 1, 0.5 to 0.8, or 0.6 to 0.7, a molecular ratio of CH₄ to CO₂ from 1 to 2, and/or a molecular ratio of O₂ to CO₂ ranges from 0.5 to 2, 0.75 to 1.5, or 1 to 1.25.

### B. Catalytic Material and Inert Material

Catalytic material used in the context of this invention may be the same catalysts, different catalysts, or a mixture of catalysts. The catalysts may be supported or unsupported catalysts. The support may be active or inactive. The catalyst support may include MgO, Al₂O₃, SiO₂, or the like. All of the support materials can be purchased or be made by processes known to those of ordinary skill in the art (e.g., precipitation/co-precipitation, sol-gel, templates/surface derivatized metal oxides synthesis, solid-state synthesis, of mixed metal oxides, microemulsion technique, solvothermal, sonochemical, combustion synthesis, etc.). One or more of the catalysts can include one or more metals or metal compounds thereof. Catalytic metals include Li, Na, Ca, Cs, Mg, La, Ce, W, Mn, Ru, Rh, Ni, Pt. Non-limiting examples of catalysts of the invention include La on a MgO support, Na, Mn, and La₂O₃ on an aluminum support, Na and Mn oxides on a silicon dioxide support, Na₂WO₄ and Mn on a silicon dioxide support, or any combination thereof. Non-limiting examples of catalysts that promote oxidative coupling of methane to produce ethylene are Li₂O, Na₂O, Cs₂O, MgO, WO₃, Mn₃O₄, or any combination thereof. Non-limiting examples of catalysts that promote dry reforming of methane to produce synthesis gas include Ni on a support, Ni in combination with noble metals (for example, Ru, Rh, Pt, or any combination thereof) on a support, Ni and Ce on a support, or any combination thereof. A non-limiting example of a catalyst that promotes oxidative coupling of methane and CO₂ reforming of methane is a catalyst that includes metals of Ni, Ce, La, Mn, W, Na, or any combination thereof. A non-limiting example of a mixture of catalysts is a catalyst mixture that include a supported catalyst containing Ni, Ce and La, and another supported catalyst containing Mn, W, and Na. The catalysts of the present invention may be layered to promote oxidative coupling in one portion of a reactor system and dry reforming of methane in another portion of the reactor. In some instances, the catalysts that promote oxidative coupling and dry reforming of methane are mixed in a desired ratio to obtain a selected amount of heat for the endothermic dry reforming reaction.

The inert material may be one or more chemically inert compounds and/or non-catalytic compounds. Non-limiting examples, of the inert material include, for example, MgO, SiO₂, quartz, graphite, or any combination thereof. The inert material can have any size or shape (for example, spheres, tubes, conical, planar, and the like) that is suitable for layering between the catalytic material. The inert material can have the same or different particle size and/or surface area as the catalytic material. The inert material does not include inert gases (for example, argon, nitrogen or both) used as in the process. In one aspect, the inert material has substantially little to no catalytic activity for oxidative coupling of methane and/or the oxidative reforming of methane. Heat generated from the oxidative coupling of methane transferred away from the catalytic material by the inert material. The heat may be removed through heat transfer from the inert material to the walls of a vessel. The inert material can be layered between catalytic material layers, mixed with the catalytic material and/or dispersed in the catalytic material. A portion of the heat generated from the oxidative coupling reaction can be removed by the inert material in amount to reduce thermal deactivation of the catalytic material.

### C. Process

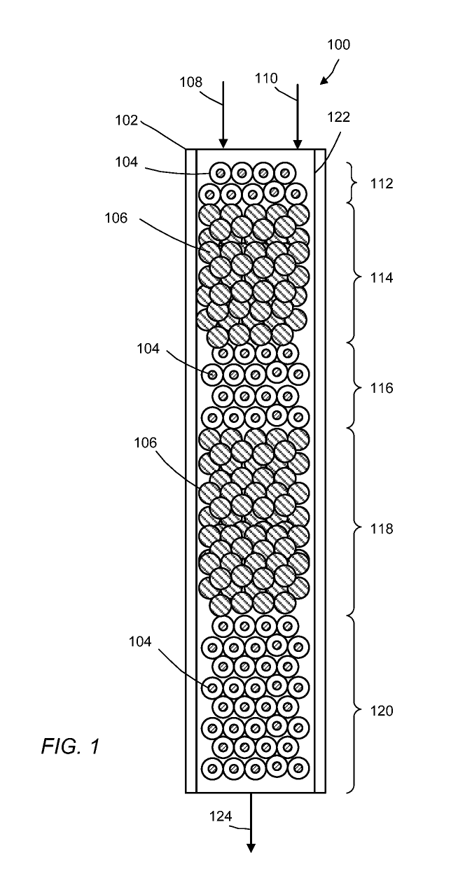

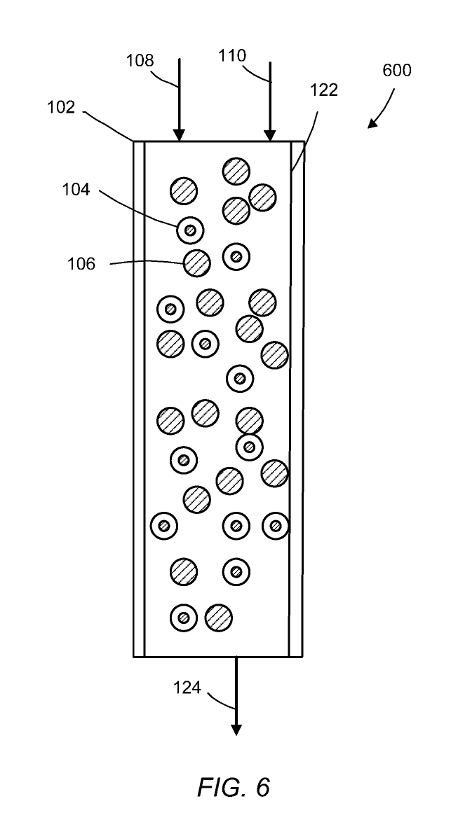

Continuous flow reactors can be used in the context of the present invention to treat methane with carbon dioxide and oxygen to produce ethylene and synthesis gas. Generally, the ethylene is obtained for oxidative coupling of methane and the synthesis gas is obtained from reforming of methane. Sufficient heat is generated to drive the endothermic dry reforming methane reaction. Non-limiting examples of the configuration of the catalytic material and the inert material in a continuous flow reactor are provided below and throughout this specification. The continuous flow reactor can be a fixed bed reactor, a stacked bed reactor, a fluidized bed reactor, or an ebullating bed reactor. In a preferred aspect of the invention, the reactor is a fixed bed reactor. The catalytic material and the inert material can be arranged in the continuous flow reactor either as separate layers in the reactor or mixed together (i.e., the catalytic material is dispersed in the inert material). Non-limiting examples of the configuration of the layers in the continuous reactor (FIGS. 1, 2 and 4) are provided below. A non-limiting example of the catalytic material dispersed in the inert material (FIG. 6) is also provided. Non-limiting examples of catalytic material and inert material that can be used in the context of the present invention are provided throughout this specification.

FIG. 1 is a schematic of system 100 for the production of ethylene and synthesis gas. System 100 may include a continuous flow reactor 102, a catalytic material 104, and an inert material 106. A reactant stream comprising methane enters the continuous flow reactor 102 via the feed inlet 108. An oxygen source and carbon dioxide are provided in via oxidant source inlet 110. In some aspects of the invention, the three reactants are fed to the reactor via separate inlets. Methane, carbon dioxide and oxygen can be provided to the continuous flow reactor 102 such that the reactants mix in the reactor to form a reactant mixture prior to contacting the first catalytic layer. The catalytic material 104 and the inert material 106 may be layered in the continuous flow reactor 102. As shown in FIG. 1, a first layer 112 of the catalytic material 104 is thin, for example, about 2-5 catalyst pellets in thickness. A first layer 114 of the inert material 106 that is thicker than the first catalytic material layer 112, for example, about 5 times thicker is positioned downstream of the catalytic material layer. A second catalytic material layer 116 is positioned downstream of the first inert material layer 114. The second inert material layer 114 is about twice the thickness of the first catalytic material layer 112, for example, 6, 7, 8 or 10 catalyst pellets in thickness. A second inert material layer 118 is about 2 times thicker than the second catalytic material layer 116, for example about 30, 40, or 50 pellets thick, and is placed downstream of the second catalytic material layer 116. A third catalytic material layer 120 fills the remainder of the continuous flow reactor 102. Contact of the reactant mixture with the first layer catalytic material 112 produces a product stream (for example, ethylene and synthesis gas (carbon monoxide and hydrogen) and generates heat (i.e., an exotherm or rise in temperature is observed). Wishing not to be bound by theory, it is believed that the product stream from contact of the feed stream with the catalytic material in the presence of oxygen generates only a small amount of carbon dioxide, due to the presence of excess carbon dioxide in the reactor. The generation of heat after contact with the catalytic layers drives the carbon dioxide reforming of methane to synthesis gas as the feed stream flows through the continuous flow reactor. A portion of the generated heat after contact with the catalytic layers is transferred to the inert layer 114, which can then transfer the heat to the walls of the reactor and/or to cooling jacket 122. The cooling jacket 122 can include one or more heat transfer fluids (for example, water, air, hydrocarbons or synthetic fluid) that can facilitate removal of heat in a controlled manner. In some instances of the invention, the continuous flow reactor 102 can include internal cooling coils, a heat exchange system or other types of heat removal components. The product stream containing ethylene and synthesis gas can exit continuous flow reactor 102 via product outlet 124.

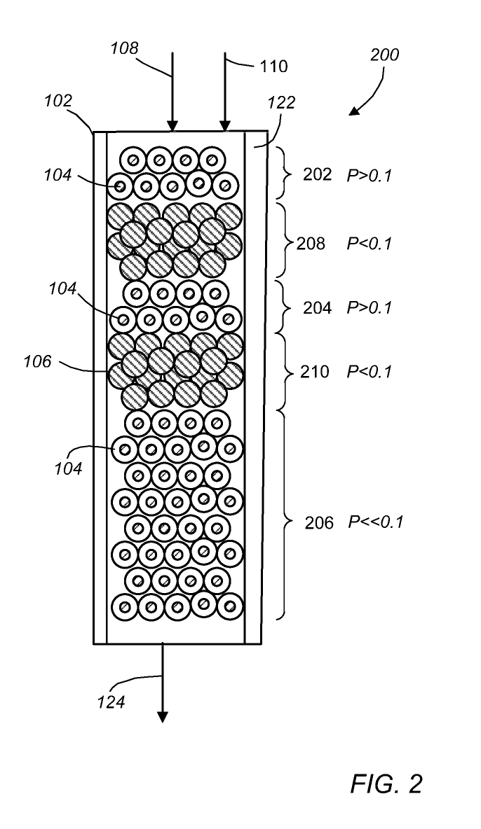

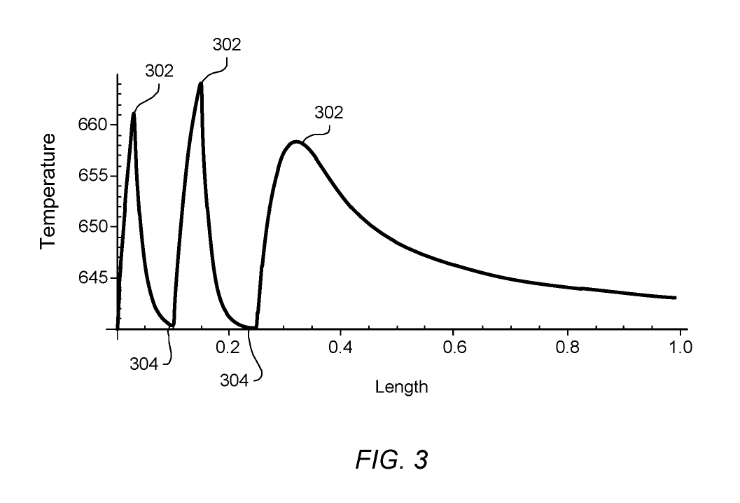

Referring to FIG. 2, FIG. 2 is a schematic of system 200 for the production of ethylene and synthesis gas that can include the continuous flow reaction 102, the catalytic material 104, the inert material 106, and the cooling jacket 122 (such as those used in system 100 for the production of ethylene and synthesis gas). Similar to system 100, the catalytic material 104 and the inert material 106 of system 200 are layered, however, the thickness of the layers are different than those shown for system 100. As shown in system 200, a first catalytic material layer 202 and a second catalytic material layer 204 are about the same thickness (for example, about two catalyst pellets thickness) and a third catalytic material layer 206 fills the remainder of continuous flow reactor 102. The catalytic layers 202, 204 and 206 are separated by inert layers 208 and 210 that are thicker than the first catalytic material layer 202 and the second catalytic material layer 204, but thinner than the third catalytic material layer 206. As shown in FIG. 2, P is less than 0.1 (P<0.1) in the inert layers 208 and 210, and P is greater than 0.1 (P>0.1) in the catalytic material layers 202 and 204. P is much less than 0.1 (P<<0.1) in catalytic layer 206. Catalytic layer 206 is used to convert the last small increment of reactants. When P is greater than 0.1 (P>0.1), the transport rate between the fluid and the catalyst limits the temperature rise in the catalyst phase, which decreases coking (or other deactivation) of the catalyst and produces more ethylene and synthesis gas instead of carbon dioxide. FIG. 3 is a graphical depiction of reaction temperature versus length of the continuous flow reactor for contact of the reactant mixture having the configuration of catalytic material layers and inert material layers described for system 200. As shown in FIG. 3, the temperature profile increases rapidly (data 302) when the feed contacts the catalytic material (P>0.1), and the temperature decreases rapidly (data 304) when the mixture of reactant mixture and product stream contact the inert material 106 (P<0.1) and heat is removed from the system. As the mixture of feed stream and product stream flow through the catalytic material layers 202, 204 and 206 along the length of the continuous flow reactor 102, the temperature profile becomes more constant as the mixture of product stream and feed stream becomes enriched in product (e.g., enriched in ethylene, carbon monoxide and hydrogen). The product stream composed of ethylene and synthesis gas can exit continuous flow reactor 102 via product outlet 124.

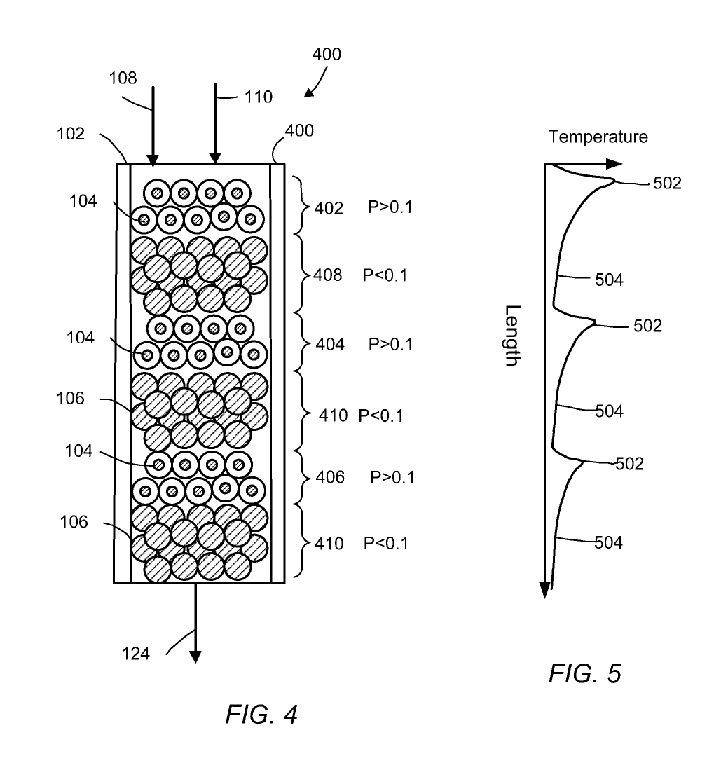

Referring to FIG. 4, a schematic of system 400 for the production of ethylene and synthesis gas that can include the continuous flow reaction 102, the catalytic material 104, and the inert material 106 (such as those used in systems 100 and 200 for the production of ethylene and synthesis gas) is described. Similar to systems 100 and 200, the catalytic material 104 and the inert material 106 of system 400 are layered, however, the thickness of the layers are different than those shown for systems 100 and 200. As shown in system 400, the first catalytic material layer 402, the second catalytic material layer 404, and the third catalytic layer 406 are about the same thickness (for example, about two catalyst pellets thickness). The catalytic material layers 402, 404, and 406 are separated by the inert material layers 408 and 410 that are substantially thicker than the catalytic material layers, for example about 10 times as thick. FIG. 5 is a graphical depiction of reaction temperature versus length of the continuous flow reactor for system 400. As shown in FIG. 5, the temperature profile small increases in temperature (data 502) occurs when the feed contacts the catalytic material (P>0.1), and a less rapid decrease in temperature is observed (data 504) as the inert material removes heat (P<0.1) from the system in a controlled manner as the feed stream and product stream flow through continuous flow reactor 102. The product stream composed of ethylene and synthesis gas can exit continuous flow reactor 102 via outlet 124.

In some aspects of the present invention, the catalytic material is dispersed in or mixed with the inert material. FIG. 6 depicts system 600 for the production of ethylene and synthesis gas that has the catalytic material 104 mixed with the inert material 106.

The resulting syngas, water, and ethylene produced from the systems of the invention (for example, systems 100, 200, 300 and 400) are separated using gas/liquid separation techniques, for example, distillation, absorption, membrane technology to produce a gaseous stream that includes carbon monoxide, hydrogen, ethylene product, and a water stream. The ethylene is separated from the hydrogen and carbon monoxide using gas/gas separation techniques, for example, a hydrogen selective membrane, a carbon monoxide selective membrane, or cryogenic distillation to produce, ethylene, carbon monoxide, hydrogen or mixtures thereof. The separated or mixture of products can be used in additional downstream reaction schemes to create additional products or for energy production. Examples of other products include chemical products such as methanol production, olefin synthesis (e.g., via Fischer-Tropsch reaction), aromatics production, carbonylation of methanol, carbonylation of olefins, the reduction of iron oxide in steel production, etc. The method can further include isolating and/or storing the produced gaseous mixture or the separated products.

### D. Conditions

The reaction processing conditions in the continuous flow reactor 102 can be varied to achieve a desired result (e.g., ethylene product and/or synthesis gas production). The method includes contacting a feed stream of hydrocarbon and oxidant (oxygen and carbon dioxide) with any of the catalysts described throughout the specification under sufficient conditions to produce hydrogen and carbon monoxide at a ratio of 0.35 or greater, from 0.35 to 0.95, or from 0.6 to 0.9 and ethylene. Such conditions can include a temperature range of 700 to 900° C. or a range from 725, 750, 775, 800, to 900° C., or from 700 to 900° C. or from 750 to 850° C., a pressure of about 1 bara, and/or a gas hourly space velocity (GHSV) from 1800 to 80,000 h⁻¹, preferably from 1800 to 50,000 h⁻¹, or more preferably from 1,800 to 20,000 h⁻¹. Severity of the process conditions may be manipulated by changing, the hydrocarbon source, oxygen source, carbon dioxide source, pressure, flow rates, the temperature of the process, the catalyst type, and/or catalyst to feed ratio. A process in accordance with the present invention is carried out at atmospheric pressure but using pressures more than atmospheric should not have negative effect to the conversion of methane because the reaction at the above mentioned conditions is not regulated by thermodynamic equilibrium where pressure may have significant effect.

### EXAMPLES

The present invention will be described in greater detail by way of specific examples. The following examples are offered for illustrative purposes only, and are not intended to limit the invention in any manner. Those of skill in the art will readily recognize a variety of noncritical parameters which can be changed or modified to yield essentially the same results.

### Example 1

### Production of Ethylene and Synthesis Gas from Methane, Oxygen and Carbon Dioxide Using Random Dilution

A fixed bed catalyst reactor was filled with a catalyst that was a mixture of Na₂O, Mn₂O₃, WO₃, and La₂O₃. The catalyst bed was diluted with inert quartz particles having the same particles size of the catalyst (about 20-50 mesh) at an inert material to catalyst ratio of 4. The reactor was heated to about 870° C. and a mixture of methane (CH₄), oxygen (O₂) and carbon dioxide (CO₂) in a CH₄:O₂:CO₂ ratio of 1:0.5:1 was fed to the reactor at a gas hourly space velocity of 3600 h⁻¹. The methane conversion was 50% with selectivity to ethylene at 33% and selectivity to carbon monoxide at 67%. Methane conversion was calculated using internal standard (argon) on the basis of difference of inlet and outlet concentrations of methane. Selectivity was calculated also using internal standard on the basis of concentrations of C2 products in comparison all the converted amount of methane.

### Example 2

### Production of Ethylene from Methane and Oxygen

The experiments in this example were carried out at conditions of Example 1, except that the feed was a mixture of CH₄:O₂ in a 4:1 ratio. Conversion of methane was 35% with the selectivity to ethylene at 65%, the selectivity to CO at 5%, and the selectivity to CO₂ at 30%.

When comparing Examples 1 and 3, the selectivity of ethylene was higher in Example 6 while and the selectivity to CO was higher in Example 1. It is believed that the excess CO₂ used in Example 1 reacted with methane to produce the reformation product of CO.

### Example 3

### Production of Ethylene and Synthesis Gas from Methane, Oxygen and Carbon Dioxide Using Random Dilution

A fixed bed catalyst reactor was filled with a catalyst that was a mixture of Na₂O, Mn₂O₃, WO₃, and SiO₂. The catalyst bed was diluted with inert quartz particles having the same particles size of the catalyst (about 20-50 mesh) at an inert material to catalyst ratio of 4. The reactor was heated to about 775° C. and a mixture of methane (CH₄), oxygen (O₂) and carbon dioxide (CO₂) in a CH₄:O₂:CO₂ ratio of 1:0.5:1 was fed to the reactor at a gas hourly space velocity of 2168 h⁻¹. The methane conversion was 30.0% with selectivity to C₂+at 80.3% and selectivity to carbon monoxide at 15.2% and selectivity to carbon dioxide at 4.5%. Methane conversion was calculated using internal standard (neon) on the basis of difference of inlet and outlet concentrations of methane. Selectivity was calculated also using an internal standard on the basis of concentrations of C₂+ products in comparison all the converted amount of methane.

### Example 4

### Production of Ethylene from Methane and Oxygen

The experiments in this example were carried out at conditions of Example 3, except that the feed was a mixture of CH₄:O₂ in a 4:1 ratio. Conversion of methane was 32.2% with the selectivity to C₂+ at 76.2%, the selectivity to CO at 10.9%, and the selectivity to CO₂ at 12.9%.

When comparing Examples 3 and 4, the selectivity of C₂+ was higher in Example 3 and the selectivity to CO was higher in Example 3 as well and the selectivity to CO₂ is lower in Example 3. It is believed that the excess CO₂ used in Example 3 reacted with methane to produce the reformation product of CO and the coupling of endothermic reaction and exothermic reaction reduces the hot spot temperature in the catalyst bed and lowers the CO₂ production.

1. A method of producing ethylene and synthesis gas from a reactant mixture comprising methane (CH₄), oxygen (O₂) and carbon dioxide (CO₂), the method comprising:
contacting the reactant mixture with a catalytic material to produce a product stream comprising ethylene and synthesis gas, wherein the ethylene is obtained from oxidative coupling of CH₄ and the synthesis gas is obtained from CO₂ reforming of CH₄,
wherein heat produced by the oxidative coupling of CH₄ is used in the CO₂ reforming of CH₄.

2. The method of claim 1, wherein the catalytic material comprises a catalyst, or a mixture of catalysts, that catalyze the oxidative coupling of CH₄ and the CO₂ reforming of CH₄.

3. The method of claim 2, wherein the mixture of catalysts includes a first catalyst that catalyzes the oxidative coupling of CH₄ and a second catalyst that catalyzes the CO₂ reforming of CH₄.

4. The method of claim 3, wherein the mixture of catalysts includes Na₂O, Mn₂O₃, WO₃, and La₂O₃, Na₂O, Mn₂O₃, WO₃ and SiO₂ or both.

5. The method of claim 1, wherein the ratio of CH₄:O₂:CO₂ in the reactant mixture is 1:0.5:1.

6. The method of claim 5, wherein the reaction temperature is 750° C. to 900° C.

7. The method of claim 4, wherein 20% to 60% methane was converted, and the selectivity to ethylene is 30% to 35% and the selectivity to carbon monoxide is 15% to 70%, or 65% to 70%.

8. The method of claim 1, wherein the method occurs in a continuous flow reactor.

9. The method of claim 8, wherein the continuous flow reactor is a fixed-bed reactor or a fluidized reactor.

10. The method of claim 1, wherein heat produced by the oxidative coupling of CH₄ is (1) used in the CO₂ reforming of CH₄ and (2) transferred to an inert material in an amount sufficient to reduce thermal deactivation of the catalytic material.

11. The method of claim 1, wherein the catalytic material and the inert material are configured in multiple alternating layers, and wherein the total number of layers of the catalytic material is equal to x, and the total number of layers of the inert material is equal to x−1, x+1, or x.

12. The method of claim 11, wherein the total number of layers of the catalytic material ranges from 3 to 50, 3 to 25, or 3 to 5.

13. The method of claim 12, wherein the inert layer has a thickness that is greater than the thickness of the catalytic material layer.

14. The method of claim 10, wherein the catalytic material is dispersed in the inert material.

15. The method of claim 15, wherein the ratio, by wt. %, of the catalytic material to the inert material is 5 to 30, 5 to 20, or 7 to 15.

16. The method of claim 10, wherein the inert material is magnesium oxide, silicon dioxide, quartz, or any combination thereof.

17. The method of claim 10, wherein the temperature of the catalytic material does not exceed its deactivation temperature of 800° C. to 900to ° C.

18. The method of claim 1, wherein the catalytic material comprises a catalyst, or a mixture of catalysts, that catalyzes the oxidative coupling of CH₄ and the CO₂ reforming of CH₄.

19. The method of claim 1, wherein the catalyst comprises manganese or a compound thereof, lanthanum or a compound thereof, sodium or a compound thereof, cesium or a compound thereof, calcium or a compound thereof, and any combination thereof.

20. The method of claim 19, wherein the catalyst comprises La/MgO, Na—Mn—La₂O₃/Al₂O₃, Na—Mn—O/SiO₂, Na₂WO₄—Mn/SiO₂, or any combination thereof.
